(19) United States
(12) Patent Application Publication
(10) Pub. No.: US 2026/0269039 A1
(43) Pub. Date: Sep. 10, 2026

(54) DIGITAL TWIN SYSTEMS, DEVICES, AND METHODS FOR TREATMENT OF THE MUSCULOSKELETAL SYSTEM

(71) Applicant: MAKO Surgical Corporation, Weston, FL (US)

(72) Inventors: Eric GARLING, Fort Lauderdale, FL (US); Alison LONG, Fort Lauderdale, FL (US); Amaia POLANCOS, Fort Lauderdale, FL (US); Thies WUESTEMANN, Fort Lauderdale, FL (US); Daniele DE MASSARI, Fort Lauderdale, FL (US)

(73) Assignee: MAKO Surgical Corporation, Weston, FL (US)

(21) Appl. No.: 19/662,650

(22) Filed: Apr. 29, 2026

Related U.S. Application Data
(63) Continuation of application No. 17/893,318, filed on Aug. 23, 2022.
(60) Provisional application No. 63/239,169, filed on Aug. 31, 2021.

(51) Int. Cl. G16H 20/00; A61B 5/00; G16H 10/60; G16H 15/00; G16H 40/60; G16H 50/20

(52) U.S. Cl. CPC G16H 20/00; A61B 5/6801; G16H 10/60; G16H 15/00; G16H 40/60; G16H 50/20

(57) ABSTRACT

A method for generating an electronic display of guidance for treating a target patient with a disease or disorder may comprise receiving, by one or more processors, a plurality of prior data sets, including: i) at least one data set specific to the target patient from the patient, ii) at least one data set specific to the patient from a health care service provider, and iii) at least one data set specific to a patient population with at least one common attribute with the target patient from a health care service provider; executing an algorithm, stored in a non-transitory computer-readable storage medium, to identify a pattern across the plurality of prior data sets, the pattern describing a probability of success for each of a plurality of potential treatment options for the target patient; automatically generating, by the one or more processors, a treatment pathway recommendation for the target patient.

### CROSS-REFERENCE TO RELATED APPLICATION

This application is a continuation of U.S. application Ser. No. 17/893,318, filed Aug. 23, 2022, which claims the benefit of priority under 35 U.S.C. § 119 to U.S. Provisional Patent Application No. 63/239,169, filed Aug. 31, 2021, the entirety of which is incorporated herein by reference.

### TECHNICAL FIELD

Embodiments of the present disclosure relate generally to providing guidance for treatment of musculoskeletal conditions. More specifically, the present disclosure relates to collecting and analyzing data from digital databases, patients, health care providers, surgical robots, and prior procedures in a digital twin virtual environment to develop guidance for medical treatment.

### BACKGROUND

Musculoskeletal disease presents unique problems for medical practitioners. Currently, there is no standardized approach to ensure the right treatment is given to the right patient at the right time when managing musculoskeletal conditions. Osteoarthritis is one musculoskeletal condition that accounts for a large portion of patients with musculoskeletal disease, and treatment of osteoarthritis often required joint replacement procedures.

Joint replacement procedures generally include replacing a subject's joint with prosthetic joint components. For example, a total knee arthroplasty (“TKA”) procedure includes replacement of the distal end of the femur and the proximal end of the tibia with a femoral prosthesis and a tibial prosthesis, respectively. Multiple bone resections on the distal femur and the proximal tibia are required prior to the implantations of these prostheses. Proper soft-tissue tension, joint alignment and balance are necessary for smooth and well-aligned joint movement. Without the ability to quickly access a patient's health and treatment history, a surgeon may be unable to make necessary adjustments to a joint replacement procedure to account for a patient's particular anatomy or pre-existing condition.

While implant technologies and operating procedures have improved, patient outcomes continue to vary in musculoskeletal procedures. Differing surgeon experience and skills, treatment choice, lack of perioperative care standards, patient comorbidities, and differing methods of communication and engagement with patients often results in reduced patient compliance with treatment plans and significant variability in the decisions taken by the health care professionals, which can lead to negative outcomes. In addition, inefficient patient flow through the healthcare system and limited health literacy provisions results in dissatisfied patients and avoidable revisions to treatment plans, impacting a patient's quality of life, compromising the use of resources, and increasing costs to the healthcare system.

Joint replacement is only one solution to managing musculoskeletal conditions and is regarded as a potentially avoidable end-point. A proportion of people can avoid joint replacement if they are managed using a collaborative risk assessment and treatment program, which includes other interventions such as physiotherapy, diet, and exercise to either reduce the need for surgery or at least delay it until the right time. For example, as most people with osteoarthritis are over sixty years old with a close correlation between age and onset of symptoms, they often have a multitude of associated comorbidities that must be considered effectively, such as diabetes, heart disease and obesity. There is a need to effectively track these patient characteristics throughout the patient pathway through diagnosis and treatment of musculoskeletal diseases.

There is a need to address one or more of the above-described issues with the treatment of musculoskeletal diseases and related health issues.

### SUMMARY

Embodiments of the present disclosure relate to, among other things, retrieving and analyzing information acquired throughout a patient care pathway to provide guidance to health care professionals for diagnosis and treatment of a patient. Each of the embodiments disclosed herein may include one or more of the features described in connection with any of the other disclosed embodiments.

In one example, a computer-implemented method for generating and presenting an electronic display of guidance for treating a target patient with a musculoskeletal disease or musculoskeletal disorder is disclosed. The method may comprise receiving, by one or more processors, a plurality of prior data sets, including: i) at least one data set specific to the target patient from the patient, ii) at least one data set specific to the patient from a health care service provider, and iii) at least one data set specific to a patient population with at least one common attribute with the target patient from a health care service provider; and executing an algorithm, stored in a non-transitory computer-readable storage medium, to identify a pattern across the plurality of prior data sets, the pattern describing a probability of success for each of a plurality of potential treatment options for the target patient. The method may further comprise automatically generating, by the one or more processors, a treatment pathway recommendation for the target patient; and generating and presenting an electronic display of the treatment pathway recommendation.

In other examples, the method may include one or more of the following features. The one or more processors may receive the at least one data set specific to the target patient from the patient via a web-based application. The one or more processors may receive the at least one data set specific to the target patient from the patient via a wearable electronic device. The at least one data set specific to the target patient from the patient may include a quality of life score and/or a forgotten joint score. The treatment pathway recommendation may include performing a radiological assessment or performing a magnetic resonance imaging (MRI) assessment or a computerized tomography (CT) imaging assessment. The treatment pathway recommendation may include a non-surgical treatment plan, wherein the non-surgical treatment plan includes one or more rehabilitation exercises, one or more treatments from a health care provider, and/or one or more drug treatments. The treatment pathway recommendation may include a recommended implant and a recommended joint replacement surgical plan. The recommended joint replacement surgical plan may be a full knee replacement plan or a partial knee replacement plan. The treatment pathway recommendation may include a recommended pre-surgical treatment schedule for the patient. The treatment pathway recommendation may include a recommended pre-surgical drug treatment.

In other examples, the method may include one or more of the following features. The generating and presenting an electronic display of the treatment pathway recommendation may include: displaying a probability of successful treatment for a recommended surgical treatment of the patient, and displaying a probability of successful treatment for a recommended non-surgical treatment of the patient, wherein the probability of successful treatment for a recommended surgical treatment and the probability of successful treatment for a recommended non-surgical treatment are determined using each of i) the at least one data set specific to the target patient from the patient, ii) the at least one data set specific to the patient from a health care service provider, and iii) the at least one data set specific to a patient population with at least one common attribute with the target patient from a health care service provider. The at least one data set specific to a patient population with at least one common attribute with the target patient from a health care service provider may include robot data from a plurality of prior robotic surgical procedures, wherein the robot data includes a movement of a robotic arm during a surgical procedure for each of the patients within the patient population with at least one common attribute with the target patient. The treatment pathway recommendation may include a recommended surgical plan for a robotic medical procedure, and wherein the recommended surgical plan includes a recommended movement of a robotic tool to treat the target patient. The at least one data set specific to the target patient from the patient may include post-operative data collected from the patient after a robotic surgical procedure; the at least one data set specific to the patient from a health care service provider may include data from a robotic surgical procedure conducted to treat the target patient; the at least one data set specific to a patient population with at least one common attribute with the target patient from a health care service provider may include post-operative data collected from patients who received a prior robotic surgical procedure; and the treatment pathway recommendation may include a recommended post-operative rehabilitation exercise schedule for the target patient.

In other examples, the method may include one or more of the following features. The method may further comprise executing a second algorithm, stored in a non-transitory computer-readable storage medium, to identify a pattern across the plurality of prior data sets, the pattern describing a probability of success for each of a plurality of potential treatment options for the target patient; automatically generating, by the one or more processors, an updated treatment pathway recommendation for the target patient using the probability of success for each of a plurality of potential treatment options for the target patient; and generating and presenting an electronic display of the updated treatment pathway recommendation, wherein the updated treatment pathway recommendation includes a recommended adjustment to the target patient's post-operative rehabilitation exercise schedule.

In other aspects, a system for generating and presenting an electronic display of guidance for performing medical treatment, may comprise: a computer-readable storage medium storing instructions for generating and presenting an electronic display of guidance for medical treatment; and one or more processors configured to execute the instructions to perform a method including: receiving a plurality of prior procedure data sets, wherein each prior procedure data set includes one or more of: i) at least one data set specific to the target patient and received from the target patient, ii) at least one data set specific to the target patient and received from a health care service provider, and iii) at least one data set specific to a patient population with at least one common attribute with the target patient from a health care service provider; identifying objective data identifying a predicted outcome of a medical treatment; and identifying a pattern across the plurality of prior procedure data sets, the pattern describing a characteristic of the medical treatment that achieves the patient outcome defined by the objective data. The method may further include receiving information about an instance of the medical treatment to be performed in the future to the target patient from a health care provider; automatically generating guidance for performing the medical treatment based on the characteristic identified by the pattern and the information received about the instance of the medical treatment to be performed; and generating and presenting an electronic display of the guidance for performing the medical treatment.

In other aspects, the system may include one or more of the following features. The at least one data set specific to a patient population with at least one common attribute with the target patient from a health care service provider may include robot data from a plurality of prior robotic surgical procedures, wherein the robot data includes a movement of a robotic arm during a surgical procedure for each of the patients within the patient population with at least one common attribute with the target patient. The treatment pathway recommendation may include a recommended surgical plan for a robotic medical procedure, and wherein the recommended surgical plan includes a recommended movement of a robotic tool to treat the target patient.

In other aspects, a computer-implemented method for generating and presenting an electronic display of pre-operative guidance for a surgical procedure to treat a target patient with a musculoskeletal disease is disclosed. The method may comprise receiving, by one or more processors, a plurality of prior data sets, including: i) at least one data set specific to the target patient from the patient, ii) at least one data set specific to the patient from a health care service provider, and iii) at least one data set specific to a patient population with at least one common attribute with the target patient from a health care service provider, wherein each of the patients of the patient population received the same surgical procedure as the surgical procedure for the target patient; and executing an algorithm, stored in a non-transitory computer-readable storage medium, to identify a pattern across the plurality of prior data sets, the pattern describing whether the surgical procedure for the target patient is predicted to be more difficult than the average difficulty of the surgical procedure based on the at least one data set specific to the target patient from the patient, the at least one data set specific to the patient from a health care service provider, and the at least one data set specific to a patient population with at least one common attribute with the target patient from a health care service provider. The method may further comprise automatically generating, by the one or more processors, a difficulty rating for the surgical procedure to treat the target patient; and generating and presenting an electronic display of the difficulty rating prior to the surgical procedure.

In some examples, the method may further comprise executing an algorithm, stored in a non-transitory computer-readable storage medium, to identify a pattern across the plurality of prior data sets, the pattern describing whether a portion of a surgical plan for the surgical procedure for the target patient is predicted to be more difficult than the average difficulty of the portion of the surgical procedure based on the at least one data set specific to the target patient from the patient, the at least one data set specific to the patient from a health care service provider, and the at least one data set specific to a patient population with at least one common attribute with the target patient from a health care service provider; and automatically generating, by the one or more processors, a surgical plan for the surgical procedure to treat the target patient; and generating and presenting an electronic display of the surgical plan prior to the surgical procedure, wherein the display of the surgical plan includes an indication of whether the portion of the surgical procedure is predicted to be more difficult than the average difficulty of the portion of the surgical procedure.

It may be understood that both the foregoing general description and the following detailed description are exemplary and explanatory only and are not restrictive of the invention, as claimed.

### BRIEF DESCRIPTION OF THE DRAWINGS

The accompanying drawings, which are incorporated in and constitute a part of this specification, illustrate exemplary embodiments of the present disclosure and together with the description, serve to explain the principles of the disclosure.

FIG. 1 illustrates a musculoskeletal digital twin system for providing guidance to health care professionals, according to an exemplary embodiment.

FIG. 2 illustrates a musculoskeletal digital twin data flow diagram, according to an exemplary embodiment.

FIG. 3 illustrates a process flow diagram of a musculoskeletal digital twin system, according to an exemplary embodiment.

FIG. 4 illustrates exemplary health care provider alerts provided by a digital twin system, according to an exemplary embodiment.

FIG. 5 illustrates a schematic drawing of a patient pathway, according to an exemplary embodiment.

FIG. 6 illustrates a system for providing guidance for a robotic medical procedure incorporating a digital twin system, according to an exemplary embodiment.

FIG. 7 illustrates a musculoskeletal treatment outcome prediction model, according to an exemplary embodiment.

FIG. 8 illustrates a block diagram of an exemplary digital twin architecture for processing patient data and providing guidance for patient treatment, according to an exemplary embodiment.

### DETAILED DESCRIPTION

The present disclosure is drawn to systems and methods for providing guidance for treatment of musculoskeletal diseases, among other aspects. A digital twin is a bridge between the physical and digital world that facilitates analysis of data and monitoring of systems in order to plan or predict the future. Here, the Digital Twin is a virtual representation of the biophysical aspects of a patient's musculoskeletal system, medical history, and treatment record, combined with evidence-based, artificial intelligence powered models that analyze data to provide insights to answer a range of clinical and logistics questions.

This disclosure includes systems, methods, and devices that incorporate a digital twin electronic system to store a data record of a patient's treatment journey, execute evidence-based artificial intelligence powered models that can analyze the patient data to provide answers to a range of clinical questions, and distribute the patient data and related analysis and recommendation to healthcare providers via an electronic network.

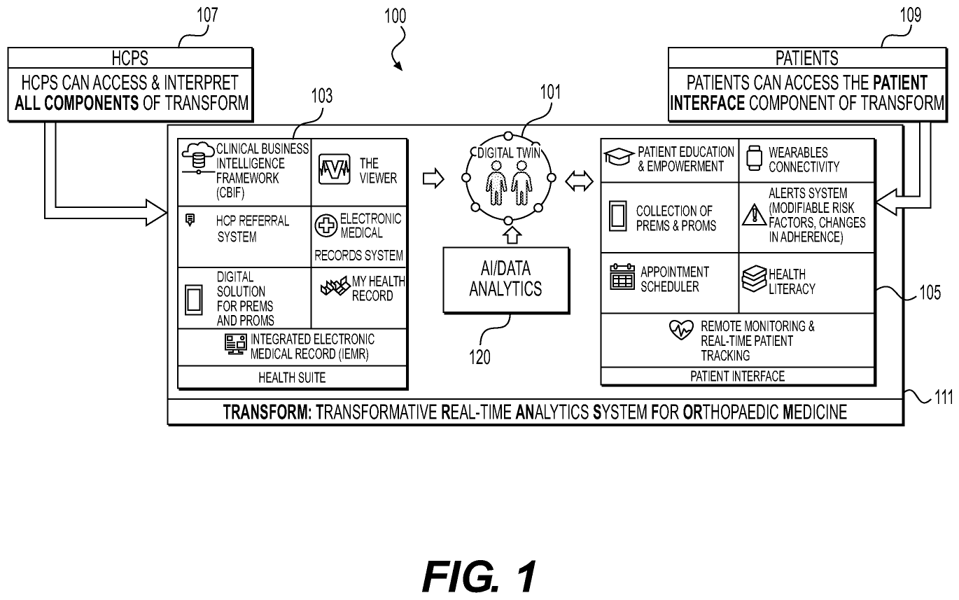

FIG. 1 illustrates a digital environment 100 including a transformative real-time analytics system for orthopedic medicine (TRANSFORM) 111 that utilizes a digital twin electronic system 101. Although discussed in the context of orthopedic medicine, the systems, methods, and devices discussed herein are not so limited and may be utilized in other healthcare context outside of orthopedic medicine. The digital twin system 101 may include an online platform provided in communication with the Internet. In some examples, the digital twin system 101 may include, for example, platform server systems and web server systems. Platform server systems may communicate with web server systems through the Internet or any other electronic network system. In one example, platform server systems and web server systems may be operated by a common owner, or fully owned and operated by a single company or entity. In another embodiment, one or both of platform server systems or web server systems may be outsourced to an Internet vendor. In some examples, a dynamic user interface may communicate with or be part of the digital twin system 101.

Environment 100 may also include health care service providers (HCSP) 107 which interact with the platform server systems and web server systems of the digital twin 101 over the Internet or other electronic network through computers, mobile devices, or other electronic devices. In some examples, health care service providers 107 may access environment 100 via a surgical robot. In general, health care service providers 107 may communicate with digital twin 101 over the Internet and may access numerous data sources 103 and artificial intelligence (AI) and data analytics 120 contained within the digital twin 101. For example, health care service providers 107 may send information about their patients, such as diagnosis or current treatment regime, to platform server systems and/or web server systems of the digital twin 101, e.g. by interacting with websites executed by web server systems or other electronic systems such as an electronic application contained within a hospital's independent electronic network. In turn, the digital twin 101 may send information about recommended patient treatments, digital models of patient anatomy or surgical environments, or other data analysis to health care service providers upon request, all through the Internet.

Environment 100, including digital twin 101, data sources 103, and patient interface 105, may implement any type or combination of computing systems, such as personal computers, mobile devices, clustered computing machines, and/or servers. In one embodiment, each computing system may be an assembly of hardware, including a memory, a central processing unit (“CPU”), and/or a user interface. The memory may include any type of RAM or ROM embodied in a physical storage medium, such as magnetic storage including hard disk or magnetic tape; semiconductor storage such as solid state disk (SSD) or flash memory; optical disc storage; or magneto-optical disc storage. The CPU may include one or more processors for processing data according to instructions stored in the memory. The function of the processor may be provided by a single dedicated processor or by a plurality of processors. Moreover, the processor may include, without limitation, digital signal processor (DSP) hardware, or any other hardware capable of executing software. The user interface may include any type or combination of input/output devices, such as a display monitor, touchscreen, keyboard, and/or mouse. Mobile devices used by health care service providers 107 or patients 109 may be configured to access wireless digital data, telephone, and/or Internet access through any other wireless communication medium, such as, for example, local or wide area Wi-Fi or Bluetooth connectivity. Mobile devices may include any type or combination of mobile phones, personal digital assistants (“PDAs”), so-called “smartphones,” tablet PC computers, or any other mobile device configured to receive communications, and display data to a user.

Digital twin 101 may be utilized to implement the various functions (e.g., calculations, processes, analyses) described herein. In some examples, digital twin 101 may include a processing circuit having a processor and memory. Processor can be implemented as a general purpose processor, an application specific integrated circuit (ASIC), one or more field programmable gate arrays (FPGAs), a group of processing components, or other suitable electronic processing components. Memory (e.g., memory, memory unit, storage device, etc.) may be one or more devices (e.g., RAM, ROM, Flash-memory, hard disk storage, etc.) for storing data and/or computer code for completing or facilitating the various processes described in the present application. Memory may be or include volatile memory or non-volatile memory. Memory may include database components, object code components, script components, or any other type of information structure for supporting the various activities described in the present application. According to an exemplary embodiment, memory may be communicably connected to processor and may include computer code for executing one or more processes described herein. The memory may contain a variety of modules, each capable of storing data and/or computer code related to specific types of functions.

It should be understood that the digital twin 101 need not be contained in a single housing. Rather, components of digital twin 101 may be located in various locations within a health care system, or even in a remote location. Components of digital twin 101, including components of the processor and memory, may be located, for example, in components of hospital or other health care providers computers, different robotic surgical systems within a healthcare environment, or outside of the health care system such as cloud based server systems.

The present disclosure contemplates methods, systems, and program products on any machine-readable media for accomplishing various operations. The machine-readable media may be part of or may interface with digital twin 101, or other aspects of environment 100. The embodiments of the present disclosure may be implemented using existing computer processors, or by a special purpose computer processor for an appropriate system, incorporated for this or another purpose, or by a hardwired system. In some examples, computer processors may be located at various discreet locations, such as within separate hospitals, within medical device company research centers, within separate countries, and/or in separate buildings of a hospital network, and each computer processing system may communicate with one another directly and/or indirectly, such as through a centralized hub. Embodiments within the scope of the present disclosure include program products comprising machine-readable media for carrying or having machine-executable instructions or data structures stored thereon. Such machine-readable media can be any available media that can be accessed by a general purpose or special purpose computer, or other machine with a processor. By way of example, such machine-readable media can comprise RAM, ROM, EPROM, EEPROM, CD-ROM or other optical disk storage, magnetic disk storage, other magnetic storage devices, solid state storage devices, or any other medium which can be used to carry or store desired program code in the form of machine-executable instructions or data structures and which can be accessed by a general purpose or special purpose computer or other machine with a processor. When information is transferred or provided over a network or another communications connection (either hardwired, wireless, or a combination of hardwired or wireless) to a machine, the machine properly views the connection as a machine-readable medium. Thus, any such connection is properly termed a machine-readable medium. Combinations of the above are also included within the scope of machine-readable media. Machine-executable instructions include, for example, instructions and data which cause a general purpose computer, special purpose computer, or special purpose processing machines to perform a certain function or group of functions.

As shown in FIG. 1, digital twin 101 may include data uploaded from health care service providers 107 and/or patients 109 through computing devices such as mobile devices or computers, and data uploaded from hospital databases and other electronic health records databases. Data sources 103 may include a Health Suite of electronic applications maintained over a network, such as a hospital network, including a Clinical Business Intelligence Framework (CBIF) which may provide data set storage, interrogation, analysis, and display of patient data. Data may include procedure information such as the general type of procedure (e.g., full or partial knee replacement; hip replacement; ankle, shoulder, or spine procedure; a procedure on a bone outside of a joint; non-orthopedic procedures, such as on soft tissue; oncology or tumor treatment procedures, such as heart procedures or orthopedic procedures; and other treatment data such as medication types, timing, and amounts). The data may also include a surgical plan with details of the procedure, such as the planned shape and order of bone modifications or which tools (e.g., saw, burr) will be used during the procedure. Data sources 103 may also include Integrated Electronic Medical Record (IEMR) applications that may store patient medical records and may serve as a replacement of paper-based clinical charts to allow healthcare professionals to simultaneously access and update patient information. In some examples, electronic medical records may be collected from several remote databases and may be compiled using one or more hospital or other health care facility electronic database. For example, vital signs and other relevant medical information of a patient may be automatically uploaded to IEMR, and may trigger early warning alerts created by digital twin 101 if a patient's condition deteriorates. My Health Record may be a web-based application that provides a national electronic health record system including a summary of an individual's important health information, including allergies, medical conditions/treatments, prescribed medications, prior surgical history, medical images, and test or scan reports. The Viewer may be a web-based application that collates data from multiple systems within a health care system network (such as a hospital's internal records system), ensuring healthcare professionals can access patients'information quickly at any stage of the patient's treatment pathway. The Viewer may be any web-based application that collects data from multiple systems within a health care system network. Digital solution for PREMs and PROMs may be a web-based application that collects and stores Patient Reported Experience Measures (PREMs) and Patient Reported Outcome Measures (PROMs). In some examples, digital solution for PREMs and PROMs may include use of software such as Philips® enterprise solution, RecoveryCOACH®, or any other web-based application that collects and stores Patient Reported Experience Measures (PREMs) and Patient Reported Outcome Measures (PROMs).

Health Care Provider (HCP) Referral System may be a web-based application that tracks health care service provider referrals and a general health care practitioner's communications with other health care service professionals. For example, HCP Referral System may provide a history of referral information for a particular health care professional and associated patient characteristics of the referred patient. Electronic Medical Records System may be a web-based application that stores electronic medical records used by a patient's general practitioner or other health care professional. Digital twin 101 may be configured to autonomously access each of the data sources 103 via an electronic network, such as the Internet or any other wired or wireless network.

Patient interface 105 can be or include wired or wireless interfaces (e.g., jacks, antennas, transmitters, receivers, transceivers, wire terminals, etc.) for conducting data communications with external sources via a direct connection or a network connection (e.g., an Internet connection, a LAN, WAN, or WLAN connection, etc.). For example, communication interfaces 28 can include an Ethernet card and port for sending and receiving data via an Ethernet-based communications link or network. In another example, communication interfaces of patient interface 105 can include a Wi-Fi transceiver for communication via a wireless communications network. Thus, if the patient interface 105 is physically separate from other components of environment 100 shown in FIG. 1, such as the digital twin 101, the patient interface 105 can enable wireless communications between the patient interface 105 and other components of environment 100 such as digital twin 101. In some examples, patient interface 105 may be a web-based application accessible via a computer, handheld electronic device, wearable electronic device, or other electronic system.

Patient interface 105 may be accessed by a patient through a computer, hand held electronic device such as a cell phone or portable computer, a wearable electronic device such as a smart watch, or other electronic devices known in the art. Patient interface 105 may provide access to patient education and empowerment features, such as information regarding a patient's care plan or reminders about rehabilitation exercises. Patient education and empowerment features may include personalized education provided through patient interface 105, which may be accessed and edited by health care providers 107 throughout treatment. Patient interface 105 may be connected to wearable electronic devices, such as a heart rate monitor or range of motion detector, to collect real-time, and other, data related to patient medical parameters and digital twin 101 may collect this data through patient interface 105. For example, a health care provider may monitor a patient's rehabilitation progress by monitoring the patient's reported range of motion electronically provided to the health care provider through the patient interface 105, which may be accessible to the health care provider through digital twin 101. Patient interface 105 may also include data collection features that allow a patient to input Patient Reported Experience Measures (PREMs) and Patient Reported Outcome Measures (PROMs). For example, a patient may input a quality of life score, a forgotten joint score, or various knee or hip scores. A quality of life score may be a numerical representation of the patient's quality of life, and a forgotten joint score may be a numerical representation of the patient's ability to forget about a joint as a result of a successful treatment. In some examples, a patient's quality of life score may be a score between 1 and 10 (inclusive), with 1 indicated a very low quality of life and 10 indicating a very high quality of life. In some examples, the patient may provide a pain score gauging the amount of pain the patient is currently feeling. Healthy patient data may be collected from a pool of patients over time and compared to injured patient data, and results of patient rehabilitation may be compared with healthy patient data to determine success or gauge progress of patient rehabilitation. In some examples, healthy patient data may be compared with the current patient data to determine changes in motion of a joint as the result from an injury, disease, exercise, and/or aging. The total number of re-admissions to a health care facility, such as the total including hospital, rehabilitation center, primary care physician's office, patient out clinic, and/or other health care facilities, may be recorded in digital twin 101, via patient interface 105 and other data sources described herein.

In some examples, patient interface 105 may track and/or record 1) the amount of time a patient takes to return to work or return to their occupation, 2) activity of daily living (ADL) (e.g. eating habits including number of meals and amount food per day, bathing habits such as number of baths a day, ability to brush teeth, ability to complete daily tasks such as grocery shopping or driving a car without assistance, etc.), 3) number of times the patient is re-admitted into a treatment facility (e.g. hospital in general, emergency room, and/or primary care facility, etc.), 4) number of rehabilitation sessions, 5) number of interactions with a health care provider (HCP), 5) fulfillment of pre-operative patient expectations such as preoperative tasks for the patient to complete, and/or 6) compliance (e.g. satisfactory or not satisfactory) completion of a functional assessment of a knee joint or other portion of the body in a gait laboratory or other facility, among other patient data.

Patient interface 105 may also include an alert system that may alert a patient if the patient is exhibiting a modifiable risk factor or change in adherence to treatment plan, or may alert the patient if the patient is exhibiting a non-modifiable risk factor that may require urgent, or other, intervention by a health care provider. For example, the patient interface 105 may be accessed through a patient's cell phone, and the cell phone may vibrate if the patient fails to input completion of a rehabilitation exercise within a scheduled period of time. In some examples, patient interface 105 may provide a reminder to the patient when an appointment with a health care provider is approaching or for a patient to exercise as part of a treatment plan. In some examples, a health care provider may modify a patient's treatment plan by electronically accessing the patient interface 105, for example if the patient has reported a new risk factor such as high blood pressure or reduced range of motion. Patient interface 105 may provide health care providers 107 with real-time data related to whether modifiable risk factors are in-fact being modified by patient activity. In some examples, patient interface 105 may include a live chat feature that allows a health care provider to directly connect with the patient via a video chat or online chatroom within patient interface 105. In some examples, the patient may interact with a software program providing a chatbot to interact with the patient via the live chat. Patient interface 105 may also enable a patient to schedule appointments with a health care service provider 107 and may allow health care service provider 107 to send a referral to the patient electronically through the patient interface 105. In some examples, patient interface 105 may display future-state treatment outcomes, including positive and negative effects of different treatment pathways and activities. For example, patient interface 105 may display an expected recovery time after a surgery if the patient effectively completes a rehabilitation program and/or may display an estimated delay in recovery time when the patient is detected to deviate from a prescribed rehabilitation program. Digital twin 101 may detect when a patient has deviated from a rehabilitation program if the patient fails to update patient interface to indicate completion of one or more prescribed rehabilitation exercises within a scheduled time period. In some examples, digital twin 101 may detect a change in activity, based on patient reported data within patient interface 109 or through data sensed via one or more remote devices (such as a sensored knee implant or wearable device), and may indicate the potential for an injury occurrence or other deteriorating effect to the patient that may be unrelated to the treatment in process, such as an ankle injury after a knee replacement.

All data collected through patient interface 105 may be sent to digital twin 101 and stored within digital twin 101. In some examples, future-state treatment outcomes may include predicted bone positioning after completion of a surgical procedure, predicted range of motion after surgery, predicted pain level (e.g. a pain score ranging from 0-10, etc.), predicted length of rehabilitation, predicted number of follow-up visits with a health care provider, and/or other common patient metrics. The future-state treatment outcomes may be displayed by patient interface 109 or any other display, such as a health care providers computer system that has electronic access to the digital twin 101 system.

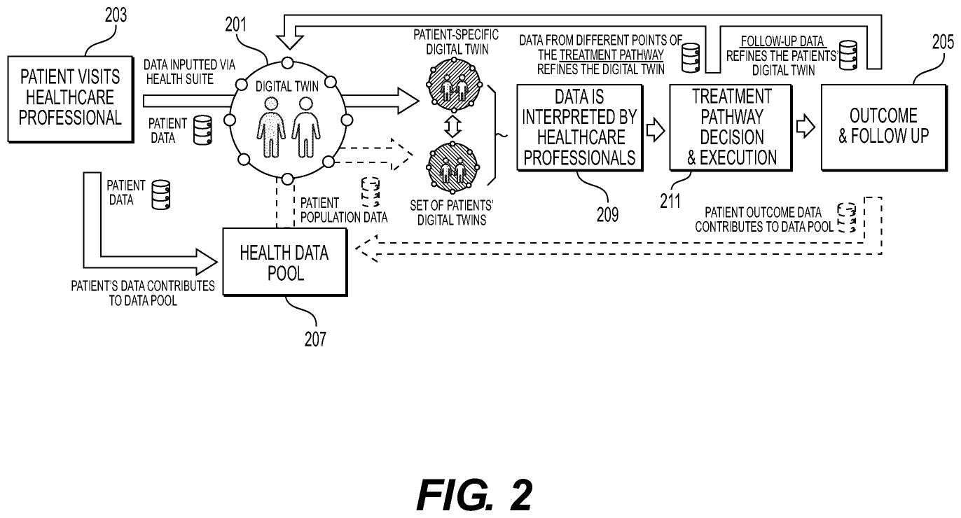

FIG. 2 illustrates a data flow diagram of a digital twin electronic system 201. Digital twin electronic system 201 may have any of the features of digital twin 101 described herein. As shown in FIG. 2, digital twin electronic system 201 collects data from a patient through the patient care pathway from the patient's initial visit to a health care professional 203 to the resulting outcome and patient follow-up 205 of the patient's treatment. In some examples, digital twin 101 may provide a health care professional, through an electronic interface, options for different joint alignments for a total knee replacement surgery, different surgery setting options (such as inpatient versus outpatient), different surgeon profile options for recommended surgeons to complete a surgery, among other treatment options. Examples of a resulting outcome include a range of motion of a joint, joint alignment after a surgical procedure or other patient treatment from a health care provider, ability to resume daily routine activities,

Patient data may be uploaded to digital twin 201, and then compared with patient population data from a health data pool 207, such as patient data collected from the health suite shown in FIG. 1. Within digital twin 201, artificial intelligence and machine learning algorithms may create models of the patient's anatomy and potential treatment regimes based on the patient's data inputted via Health Suite and the patient population data from the health data pool 207. Digital twin 201 may include virtual models of the patient's anatomy such as a model of the patient's knee (e.g. a biomechanical model), recommended courses of action for a health care provider, and databases of a patient's medical history. The patient's digital twin may be compared with a database of other digital twins of other patients, such as digital twins of other patients with one or more similar phenotypes, such as symptoms and/or anatomies/anatomical features, as the patient's symptoms or one or more similar prescribed treatments. By comparing the patient's digital twin 201 with digital twins of other patients with similar attributes as the current patient, the patient's digital twin 201 may provide risk assessment, prediction of disease development, and/or recommended courses of action for a health care provider through algorithm execution within digital twin 101. Similar attributes may include one or more attributes, such as size of joint, bone alignment, height, weight, age, portions of medical history, degree of injury, etc. Once the data from digital twin 201 is interpreted by healthcare professionals, the healthcare professionals may make a treatment pathway decision 211 and execute the next course of action for treatment of the patient. A treatment pathway decision 211 may be directing the patient to have a surgical procedure or directing the patient to proceed with non-surgical treatment, or may change one or more aspects of the surgical procedure or non-surgical treatment. In some examples, the treatment pathway decision 211 may include a recommended surgical implant or recommended surgical plan. This treatment pathway decision 211 may be uploaded and saved in digital twin 201.

After completion of the treatment pathway, the outcome data of the patient's treatment may then be uploaded and stored within digital twin 201. In some examples, a treatment pathway decision 211 may include a pre-treatment or pre-operative decision, or a post-operative or post-treatment decision. In some examples, treatment pathway decision 211 may be selecting an appropriate surgical implant or surgical plan for the patient. In some examples, a post-operative or post-treatment decision may include a recovery goal of an expected healthy state of a knee joint or other portion of the patient's anatomy based on outcome data from prior surgical procedures of patient's with one or more attributes in common with the current patient. In some examples, digital twin 101 may determine an expected impact of a surgical procedure, or the expected impact of using different implant options for a joint replacement surgery, on the patient, such as expected post-operative pain levels, expected post-operative recovery time, expected increase or decrease in range of motion of a joint, etc. In some examples, digital twin 101 may display a recommendation to proceed immediately or wait to have a recommended surgical procedure. In some examples, one or more wearable sensors may monitor a patient's weight, average heart rate, range of motion, or other physical parameters, and the digital twin 101 may receive these measured parameters and determine potential changes to rehabilitation and/or other treatment to decrease total recovery time.

In some examples, treatment pathway decision 211 may include selecting a sensored surgical implant, such as an implantable sensor or a sensored prosthetic component, that may track data of the patient post-operatively. A sensored surgical implant may be a knee implant including one or more of a motion sensor, an accelerometer, a temperature sensor, a load sensor, a vibration sensor, a range of motion detector, a camera such as a video camera, or other sensor positioned within or coupled to any portion of the knee implant. In some examples, a sensored implant may communicate wirelessly with digital twin 101 and may transmit data to digital twin 101, directly or indirectly. For example, treatment pathway decision 211 may include recommending a sensored knee implant that may collect data, such as range of motion, load applied to the implant, movement of the implant, monitoring infection or other characteristics of the body in the area of the body surrounding the implant, or other parameters. Data collected by a sensored implant may be uploaded to digital twin 101 and may be used to optimize future procedures or other recommendations for the patient or other future patients. Sensored implant data may be used post-operatively to recommend post-operative treatments to the patient, such as an adjustment to a rehabilitation exercise program or a recommended additional medical procedure or a recommended follow-up visit to a health professional.

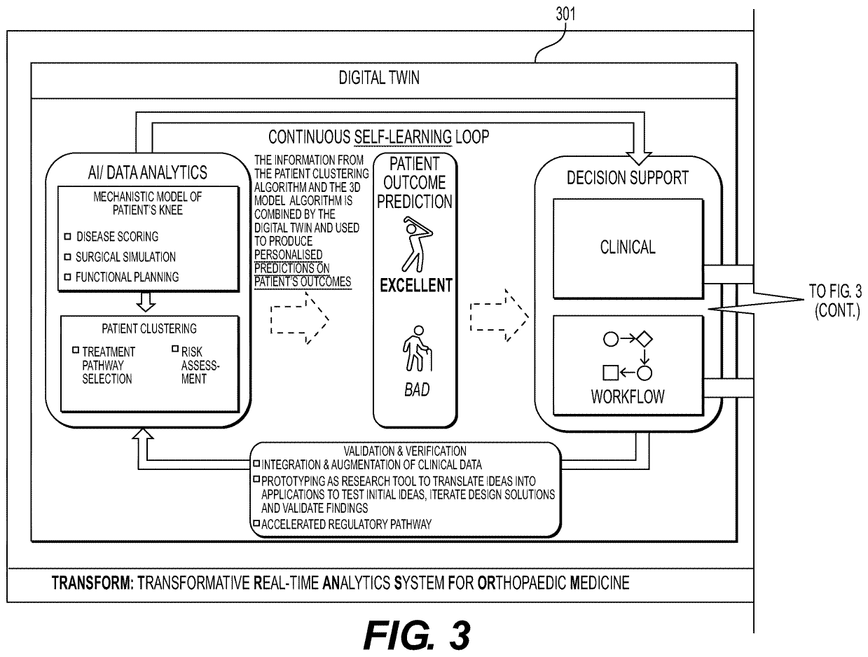

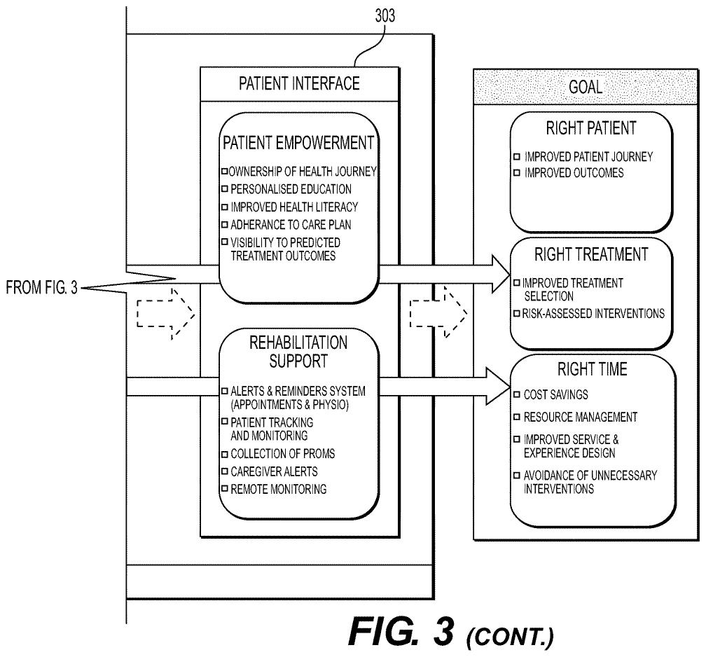

FIG. 3 illustrates a data flow diagram between a digital twin 301 and a patient interface 303. Digital twin 301 and patient interface 303 may have any of the features described herein related to digital twins 101, 201 and patient interface 105. As shown in FIG. 3, digital twin 301 may provide clinical decision support to health care service providers and provide workflow optimization support within a health care facility. Artificial intelligence and data analytic algorithms within digital twin 301 may be configured to continuously execute feedback loops collecting data from the patient and health care providers throughout the patient pathway. By allowing a continuous stream of patient data and health care provider data inputted into digital twin 301, digital twin 301 may continuously learn based on the results of prior procedures and current, real-time results of treatment to the target patient. Digital twin 301 may then adjust recommendations for treatment or other actions based on real-time patient data and health care provider data.

In some examples, digital twin 301 may include an algorithm to determine identifying risk factors and/or a risk assessment for clinical decision support to health care service providers. For example, digital twin 301 may execute an algorithm using data from a plurality of other digital twins from patients with one or more similar attributes to determine modifiable risk factors, such as losing weight or stopping smoking. In some examples, digital twin 301 may display a patient outcome prediction if the patient adheres to modifying an identified risk factor, such as if the patient loses weight, and also displays a patient outcome prediction if the patient does not modify the identified risk factor. In some examples, digital twin 301 may provide a comorbidity assessment. In some examples, digital twin 301 may assess and/or monitor chemical levels (e.g., alcohol, sugar intake, drugs or medications) in a patient's blood stream or tissues. The digital twin 301 may monitor and/or determine medications provided before, during, or after a procedure. The digital twin 301 may also assess and/or monitor smoking, alcohol, or sugar intake and consider such factors when determining and/or optimizing aspects of a procedure plan.

Digital twin 301 may also provide mechanistic models of the patient's anatomy, such as an electronic model of a biophysical representation of the patient's knee or other musculoskeletal anatomy, such as a computer-simulated model of a patient's knee showing the current, pre-operative state of the patient's knee joint and/or a resulting alignment of bones after surgery, etc. In some examples, digital twin 301 may be configured to display the electronic model of the patient's anatomy and highlight areas of the anatomy that indicate potential problems or treatment areas. Digital twin 301 may also provide patient referrals to a specialist, disposition recommendations, diagnostic pathway recommendations such as the need for additional patient assessments, such as patient imaging assessments such as a radiological assessment, a magnetic resonance imaging (MRI) assessment, an x-ray assessment, computerized tomography (CT) assessment, other imaging assessments, and/or treatment pathway recommendations. In some examples, digital twin 301 may include an algorithm to determine a recommended knee treatment, such as recommending a total knee arthroplasty or a partial knee arthroplasty. Digital twin 301 may utilize patient population data to create a surgical simulation, a treatment plan, a surgical plan, and/or a resource allocation recommendation. In some examples, digital twin 301 may generate predicted outcomes, such as estimated rehabilitation periods of time, estimated time to return to work or estimated pain levels, to provide to the patient through the patient interface 105. Predicted outcomes displayed through digital twin 301 and/or patient interface 105 may help manage patient expectations.

In some examples, digital twin 301 may utilize data to provide a recommendation on using a surgical robotic or a robotic procedure, when to use manual steps or a procedure, and/or when to use other surgical assisting devices. For example, digital twin 301 may determine different levels of tools. Each tool may be associated with a predetermined score, which may be input as data and/or determined by digital twin 301. The digital twin 301 may determine, using data and/or by executing one or more algorithms, an assistive score for the procedure to indicate a level of intelligent or artificial intelligence (AI) involvement. For example, a determined assistive score in a first range (e.g., 0 to 20) may indicate a typical non-computer assisted surgery, a determined assistive score in a second range (e.g., 20 to 40) may indicate a procedure that uses some type of computer guidance tool, and a determined assistive score in a third range or above the second range (e.g., 40 and above) may indicate a robotic procedure or the use of an increasing number of intelligent tools. Digital twin 301 may also indicate a need for different tools at different steps of the procedure. Digital twin 301 may determine different surgical approaches or techniques for a procedure.

In some examples, predicted outcomes may be in the form of a predicted successful surgery, a predicted patient post-operative diagnosis, a predicted post-operative complication from surgery, a predicted likelihood of complications due to surgery, a predicted post-surgery pain level, a predicted bone re-alignment, a predicted recovery time, a predicted timeline for partial and/or full recovery from surgery, a predicted success of rehabilitation after surgery, a predicted timeline until a patient is able to walk again, a predicted post-operative side-effect of surgery, a predicted range of post-operative Patient Reported Experience Measures (PREMs) and/or Patient Reported Outcome Measures (PROMs), and/or a virtual model of a predicted patient's anatomy post-surgery.

In some examples, digital twin 301 may include algorithms that utilize Bayesian Learning approaches. For example, digital twin 301 may include a Model-based algorithm (e.g., Bayesian Network modelling) that can capture contextual factors related to an individual patient. A Bayesian Network would establish a directed acyclic graphical network model of the patient flow as a ‘complex system’ to capture the complex interactions within the system (e.g., the multiple factors affecting individuals as well as all participants within the system). This approach captures key factors that affect patient outcomes, as well as other factors (e.g., environmental, co-morbidities, etc), which can be used to quantify the probabilities associated with the predicted outcomes of an individual. The more data that is included will increase the predictability of the model. Digital twin 301 may also include simulation-based algorithms (e.g., Agent-based modelling), where each entity (e.g., patient) is established as an agent with characteristics. This can be useful when learning from other patients (e.g., outcomes of other patients with characteristics (e.g., age, co-morbidities, treatment regime, probability of adverse outcomes, probability of ‘good’ outcomes of treatment, expected timespan-related outcomes (short, medium longer), etc.). This approach enables learning from individual patients, as well as all patients.

In some examples, digital twin 301 may include one or more algorithms to predict the difficulty of a surgical procedure. For example, digital twin 301 may generate an expected surgical time based on prior procedure data sets and patient specific characteristics. Digital twin 301 may notify a health care service provider if a surgical procedure is predicted to be more difficult than the average surgical procedure to allow the surgeon to adjust the schedule to account for the increased difficulty of the surgery, and/or adjust resources required such as surgical tools needed or amount, type of staff (which may be outside of standard practice procedures) required for a surgery, change in a surgical workflow, and/or a change in surgical location (such as ambulatory surgery center versus hospital, etc.).

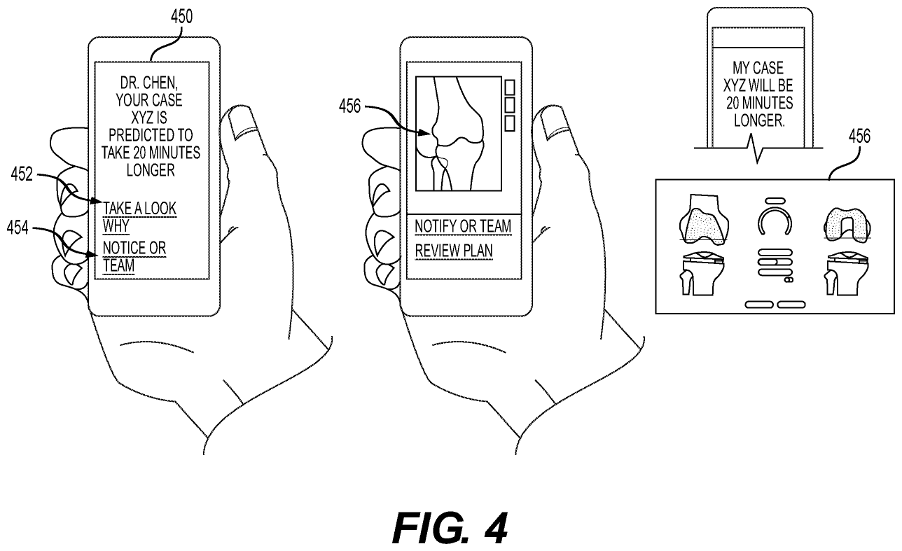

FIG. 4 illustrates examples of notifications 450 that may be generated by digital twin 101 (and/or digital twin 201 and/or 301 shown in FIGS. 2 and 3) when a surgical procedure is determined to be more difficult than a normal surgical procedure of the same type (or the anticipated level of difficult for that surgical procedure). For example, a surgeon may receive a notification 450 on his or her desktop computer and/or mobile electronic device indicating a patient's procedure is predicted to take 20 minutes longer than the scheduled amount of time for the procedure. Digital twin 101 may generate selectable options for a user to either 1) review information related to why the surgical procedure is predicted to take longer than expected 452; or 2) notify other members of an operating room team 454, such as nurses, physicians assistants, doctors, etc. regarding the predicted time extension needed for the procedure. Information regarding why the surgical procedure is predicted to take longer may be displayed electronically to a user, and notifications to other members of the operating room team 454 may be sent electronically over a network to electronic devices accessible to the other members. In some examples, digital twin 101 may generate potential reasons why the procedure may be more difficult for that particular surgeon operating on the particular patient and may display images 456 to the user related to potentially difficult regions of the patient's anatomy to operate on. In some examples, digital twin 101 may generate case-specific training material for display to the healthcare professional, such as guidance for a step of a surgical procedure such as an incision length or position, or positioning of a prosthetic implant.

In some examples, digital twin 101 may receive one or more medical images from data sources 103, and may execute an algorithm to determine whether a surgical procedure, such as a total knee or partial knee arthroplasty, is predicted to be more challenging than an average total knee or partial knee arthroplasty. If the surgery is predicted to be more difficult, digital twin 101 may recommend an adjustment of the surgical schedule, or surgery day or location, to account for additional time required for the subject surgical procedure or may automatically adjust a surgical schedule to incorporate additional time for the subject surgical procedure. The algorithm may receive additional patient specific data such as patient age, gender, height, weight, and/or body mass index (BMI). The algorithm may also receive from a health care provider, calculate using one or more medical images, or calculate using a mechanistic model of the patient's anatomy, one or more anatomical measures including: aHKA, anatomical hip-knee-ankle angle; MPTA, medial proximal tibial angle; LDFA, lateral distal femoral angle; a joint line angle such as the angle formed between a line tangential to the distal femoral condyle and the tibial plateau; etc. Digital twin 101 may determine and/or receive data on anatomical variances, such as varus/valgus joint shape, mechanical and functional axis, contact locations of joints through a range of motion, alignment of bones of a joint, disease state, soft tissue locations, bone or tissue shape, thicknesses, diameters, densities, ligament size, and/or location of tears.

Digital twin 101 may execute an algorithm that predicts the total surgical time of a procedure based on input data 103 and data received through patient interface 105. In some examples, digital twin 101 may execute an algorithm to determine whether a surgical procedure is predicted to be more difficult than an average surgical procedure of a particular type using data supplied by a surgical robot. If a robotic surgical procedure is predicted to be more difficult than average, one or more planned movements of the surgical robot may be adjusted based on the predicted difficulty of the surgical procedure. In some examples, one or more algorithms of digital twin 101 may generate a predicted difficulty score for a planned surgical procedure and indicate the degree of difficulty to a user via a numerical score, such as a score between 1 and 100 with 100 being the most difficult and 0 being the least difficult. In some examples, one or more algorithms of digital twin 101 may adjust a predicted surgical time of a patient's procedure based on surgical robot data received from a prior patient procedure with one or more patient anatomical features similar to the subject. In some examples, digital twin 101 may determine recommended tools for use during a procedure, such as recommend manual tools compared to robotic tools, such as the use of a robotic surgical system including a robotic arm. In some examples, the surgical approach an/or type of incision may be included in the surgical plan and may be adjusted relative to an average procedure of the same type.

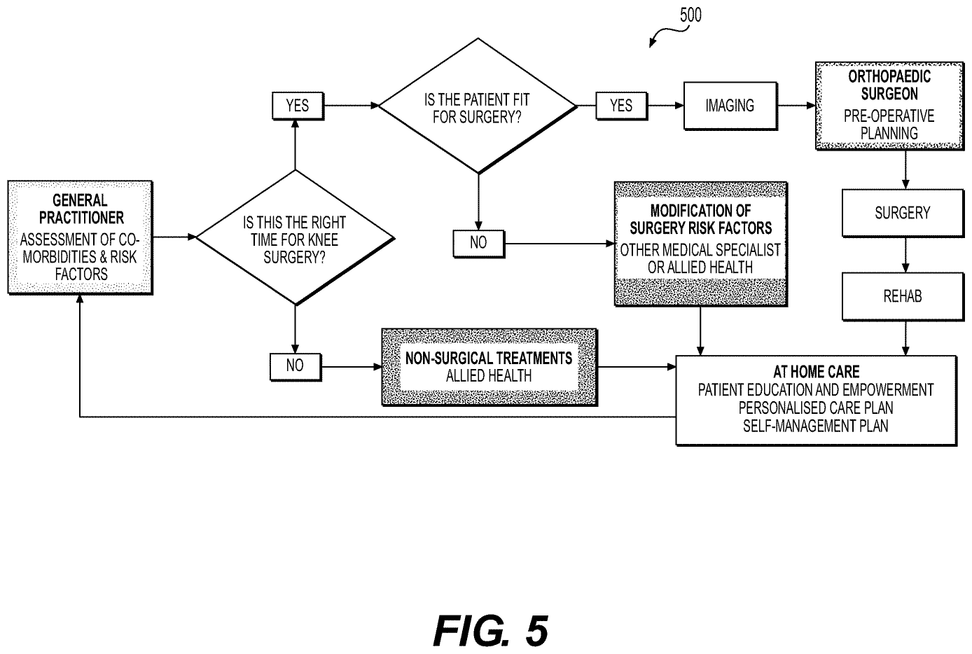

FIG. 5 illustrates an exemplary treatment pathway 500 of a patient with knee pain as a result of osteoarthritis. Similar treatment pathways may be utilized by patients with other musculoskeletal diseases. Initially the patient visits a general practitioner to assess comorbidities and risk factors of the patient that may contribute to poor treatment outcomes or make the patient unfit for surgery. In some examples, digital twin 101 may be accessed by the general practitioner to help determine whether it is the right time for knee surgery. Digital twin 101 may provide the general practitioners with electronic access to a patient's medical records and other patient data. In some examples, the general practitioner may communicate with a surgeon through digital twin 101 to facilitate the decision on whether to proceed with knee surgery (or other joint replacement surgery, or any surgery to treat musculoskeletal disease or disorders, including spine and/or trauma injuries.) If the decision is made to not proceed with knee surgery, the general practitioner may refer the patient to allied health (non-surgical) treatments such as physiotherapy or nutrition and the patient will proceed with at home care. In some examples, the general practitioner may provide an at home treatment plan to the patient through digital twin 101 and accessible to the patient through patient interface 105. The patient's progress with the at home care may be monitored through the patient interface 105 and digital twin 101. If the general practitioner decides it is the correct time for knee surgery and the patient is fit for surgery, the patient may proceed with having medical imaging done such as x-ray imaging, ultrasound, computerized tomography (CT) scan, Magnetic Resonance Imaging (MRI), or other types of medical imaging. Medical image data may be uploaded to digital twin 101 immediately after the patient has the imaging done, the medical image data may be accessible to the general practitioner and other specialists immediately through digital twin 101. After imaging, an orthopedic surgeon may access digital twin 101, which may include all of the medical images of the patient along with the general practitioners assessment of the patient, to plan for surgery. In some examples, digital twin 101 may provide the orthopedic surgeon with a recommended operative plan based on one or more algorithms that process data inputs 103. During surgery, digital twin 101 may provide the surgeon with real-time predictive models and potential problems that may arise during the surgery. In some examples, digital twin 101 may provide a surgical robot with guidance via an electronic network, along with guidance to the orthopedic surgeon prior to, during, and after the surgical procedure. After surgery, digital twin 101 may provide recommended rehabilitation plans to aid health care providers in assigning an appropriate rehabilitation plan to the patient. Digital twin 101 is not limited to assisting health care professionals during the events shown in patient pathway 500, and may be utilized in any treatment pathway for musculoskeletal diseases or related patient treatments.

In some examples, digital twin 101 may receive data about and/or determine an order of treatments. For example if a patient needs a tumor removed and a knee replacement, digital twin 101 may identify a preferred timing and sequence of steps to be completed for the desired patient outcome.

In some examples, digital twin 101 may offer alternative steps, procedures, and/or treatment pathways and provide a choice to a reviewing practitioner and/or patient. Digital twin 101 may provide and/or display options as to a desired outcome or desired treatment and provide estimates of success of each treatment or option. For example one patient may prefer to have a surgery as quickly as possible, while another may prefer more non-surgical options. Digital twin 101 may provide anticipated pain and activity levels for each option so that the patient may have an informed choice. A patient and/or practitioner may enter, as input data into Digital twin 101, medications they do not want to take (e.g., due to allergies) or types of surgical procedures they would like to avoid. These entries may be considered by the Digital twin 101 when determining alternatives. For example, digital twin 101 may restrict out some options based on a patient and/or practitioner's input as to what to avoid.

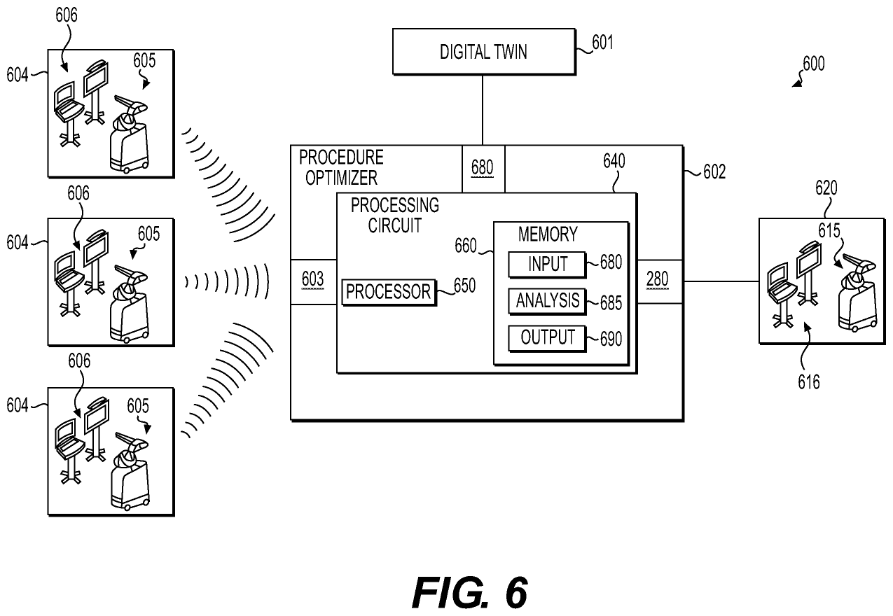

FIG. 6 illustrates a system 600 for developing guidance for robotic medical procedures utilizing a digital twin 601. The system 600 includes a procedure optimizer 602. The procedure optimizer 602 may receive input procedure data 603 from multiple robotic systems 604. In one embodiment, each set of input procedure data 603 corresponds to a completed or in-progress robotic medical procedure performed by a user using a robotic system 604. A “user” may be synonymous with “practitioner” and may be any person completing the described action (e.g., surgeon, technician, nurse, etc.). Among other components, each of the robotic systems 604 may include a robotic device 605, a guidance module, and a camera stand for tracking the patient and other objects. The guidance module and camera stand (referred to herein as guidance components 606) may include screens for providing output to a user. One or more of the robotic device 605, guidance module, or camera stand may store input procedure data 603 that may be retrieved by the procedure optimizer 602. Input procedure data 603 may alternatively be stored in any other component of the robotic system 605, or may be stored outside of the robotic system 605. In some examples, input procedure data 603 may be obtained electronically through digital twin 601. The stored input procedure data 603 may be analyzed by the procedure optimizer 602 to determine patterns of characteristics of the corresponding medical procedures. Digital twin 601 may provide procedural optimizer 602 with patient data preoperatively, intraoperatively, and postoperatively.

The procedure optimizer 602 may further receive information about an assisted robotic medical procedure through digital twin 601. The procedure optimizer 602 may then develop guidance for the robotically assisted procedure based on the analysis of input data 603 and the digital twin 601 information. An “assisted procedure” refers to a robotic medical procedure for which guidance from the procedure optimizer 602 would be relevant. The term “assisted procedure” is used herein to distinguish the procedure the guidance is directed towards, or relevant to, from the procedures and their corresponding data used as inputs to the procedure optimizer 602. The assisted procedure may or may not have been started or completed at the time the guidance is developed or provided to a user. Furthermore, the assisted procedure may be a planned procedure that never actually takes place, a procedure that is partially completed, or a procedure that has already been completed. The output provided by the procedure optimizer 602, which may be guidance relevant to the assisted procedure, may or may not actually be acted upon by a user, and if acted upon, the guidance may or may not in fact optimize the procedure with respect to a given metric. Digital twin 601 may provide procedure optimizer 602 with information from prior robotic surgical procedures collected in digital twins of other patients. Digital twin 601 may also receive robot data related to the patient's surgical procedure from surgical robot 615, guidance components 616, and/or procedure optimizer 602, and such robot data may include specific movements of the surgical robot, tools used by the surgical robot, and/or positioning of the surgical robot within the operating room. In some examples, digital twin 601 may provide instructions for movement of one or more tools of the robotic arm of robotic device 605. For example, digital twin 601 may track movement of each robotic arm of each robotic device 605 within a network of robotic systems 604, and may use the data collected associated with the movement of each robotic arm of each robotic device 605 to recommend a movement of a robotic device 615, such as a specific movement of a robotic arm, for a future robotic surgery.

The procedure optimizer 602 may include a processing circuit 640 having a processor 650 and memory 660. Processor 650 can be implemented as a general purpose processor, an application specific integrated circuit (ASIC), one or more field programmable gate arrays (FPGAs), a group of processing components, or other suitable electronic processing components. Memory 660 (e.g., memory, memory unit, storage device, etc.) may be one or more devices (e.g., RAM, ROM, Flash-memory, hard disk storage, etc.) for storing data and/or computer code for completing or facilitating the various processes described in the present application. Memory 660 may be or include volatile memory or non-volatile memory. Memory 660 may include database components, object code components, script components, or any other type of information structure for supporting the various activities described in the present application. According to an exemplary embodiment, memory 660 may be communicably connected to processor 650 and may include computer code for executing one or more processes described herein. The memory 660 may contain a variety of modules, each capable of storing data and/or computer code related to specific types of functions. In one embodiment, memory 660 contains several modules related to medical procedures, such as an input module 680, an analysis module 685, and an output module 690.

It should be understood that the procedure optimizer 602 need not be contained in a single housing. Rather, components of procedure optimizer 602 may be located in various locations, or even in a remote location. Components of procedure optimizer 602, including components of the processor 650 and memory 660, may be located, for example, in components of different robotic systems 605 or in the robotic system components (e.g., in the guidance components 506) of the assisted procedure. Procedure optimizer 602 may be connected to digital twin 601 via an electronic network such as the Internet or an internal network of a health care system. In some examples, procedure optimizer 602 may be part of digital twin 101, 601, part of a surgical robot 615, or both part of digital twin 101, 601 and one or more surgical robots, 605, 615. Any of the features of digital twin 101, 601 discussed herein may be incorporated with a robotic surgical procedure, and procedure optimizer 602 may receive recommendations from digital twin 101, 601 and may adjust a generated robotic surgical plan based on recommendations from digital twin 101, 601. In some examples, digital twin 101 may generate a predicted outcome for the subject patient of a robotic surgical procedure and also a predicted outcome for the subject patient of a non-robotic surgical procedure.

In some examples, digital twin 101 may collect data from a patient, health care provider, or other source to confirm a predicted outcome after a surgery. For example, a predicted recovery time may be confirmed by receiving patient reported outcome measures after surgery, such as pain scores, forgotten joint scores, measured range of motion of a patient's anatomy, or other data collected from the patient to confirm a predicted outcome. In some examples, digital twin 101 may collect data from a wearable electronic device of the patient to confirm a particular predicted outcome, such as data from a heart rate monitor to confirm patient's overall health or data from a wearable device detecting range of motion of a joint to confirm rehabilitation success. By confirming a predicted outcome, digital twin 101 may further optimize future recommendations for patients with one or more similar attributes to the patient tracked by digital twin 101. In some examples, digital twin 101 may collect data from exercise and/or rehabilitation equipment. Such equipment may provide information directly to digital twin 101 (e.g., through sensored equipment) or indirectly (e.g., by a health care work monitoring or tracking activity) related to a strength of a muscle and/or a range of motion or level of cardiovascular activity. Exercise equipment, such as a treadmill or exercise bike, may provide information related to activities such as distance, speed, and resistance levels to monitor patient improvements.

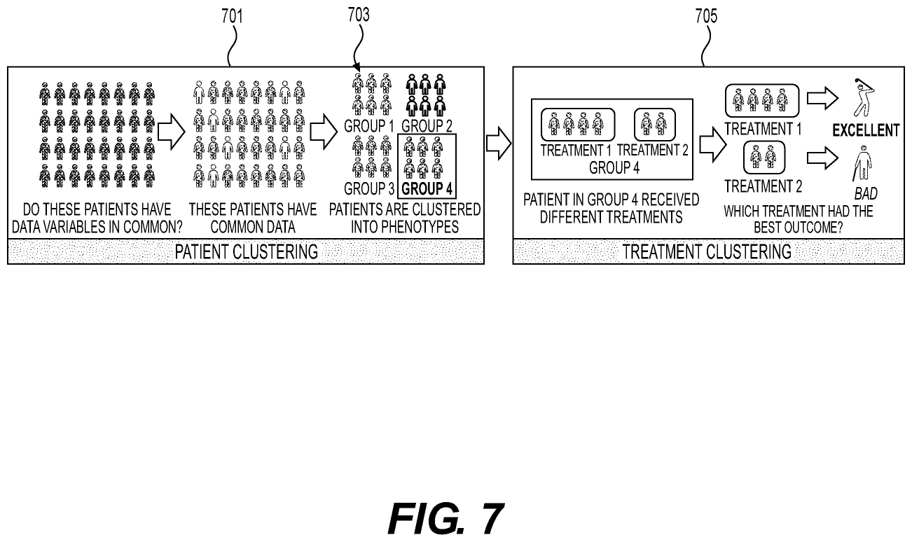

Digital twin 101 may implement algorithms that cluster patients into groups of similar patients. For example, pre-operative information may be used to cluster patients in different sub-cohorts based on a selected similarity measure (different measures can be retrieved from each patient's digital twin and applied to this use case). Initially the patient clustering 701 may be based on historical data. Each new case, or new instance of patient treatment, may be added to the digital twin dataset, so case history may influence future analysis data outputs. Each sub-cohort is considered a phenotype 703 (shown in FIG. 7). In some examples, every new patient may be assigned to a phenotype 703 and the health care service provider may be presented, through digital twin 101, with descriptive statistics referring to the selected phenotype 703 to enable informed decisions. Information such as historical average post-op outcomes, most used intra-op parameters, and average treatment success rates may be shown. Digital twin 101 may use treatment clustering 705 to track treatment outcomes of each phenotype 703 to adjust future treatment recommendations. For example, all patients in a specific phenotype may be tracked and clustered into the type of treatment received, and outcomes for each patient may be compared to facilitate future treatment selection. In some examples, a user may select specific patient clusters or treatment clusters, and view information related to specific phenotypes 703 and receive recommendations based on a patient being assigned, via digital twin, to a specific phenotype 703. In some examples, the user may select specific recommended surgeons and a profile of predicted outcomes may be displayed for each recommended surgeons for a patient to compare. Similarly, the user may select specific recommended rehabilitation protocols and a profile of predicted outcomes may be displayed for each recommended rehabilitation protocol for a patient to compare. In other examples, the user may select specific recommended implant types and a profile of predicted outcomes may be displayed for each recommended implant type for a patient to compare.

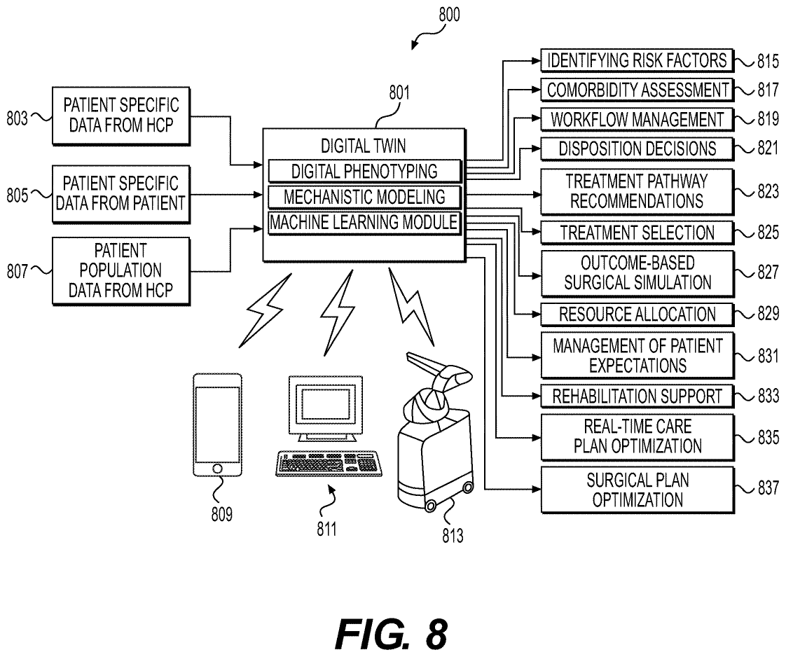

FIG. 8 illustrates a block diagram of an exemplary digital twin architecture 800. Digital twin 801 may receive patient specific data from a health care provider or other data source 803, patient specific data from the patient through a patient interface 805, and patient population data from a health care provider or other data source 807. Throughout the patient treatment pathway, digital twin 101, 601, 801 may receive data from and transmit data to electronic devices 809, computers 811, and surgical robots 813 to provide information to health care providers and/or facilitate treatment of the patient. Digital twin 801 executes digital phenotyping, mechanistic modeling, and machine learning algorithms with the received data, and may output identifying risk factors 815, comorbidity assessment 817, workflow management recommendations 819, recommended disposition decisions 821, treatment pathway recommendations 823, treatment selection recommendations 825, outcome-based surgical simulations 827, resource allocation recommendations 829, recommendations for treatment education for management of patient expectations 831, recommended rehabilitation support 833, real-time care plan optimization recommendations 835, and/or surgical plan optimization recommendations 837. In some examples, digital twin 801 may recommend a specific location for placement of an acetabular cup screw. In some examples, digital twin 801 may recommend a treatment facility (e.g. in-patient, outpatient, ambulatory surgery center), a place of discharge (e.g. patient's home residence or nursing facility), patient specific education (e.g. materials according to expected outcome/risks), and/or required case specific instruments (e.g. surgical robot, burr, screw, motorized knife, etc.).

Although partial and total knee arthroplasties are referred to herein as examples, the digital twin 101 may be used in the field of oncology to treat cancer. For example, the digital twin 101 may include procedure data or other data related to tumor treatment procedures, and aspects disclosed herein may evaluate the tumor treatment procedures or combinations of procedures (e. g, heart procedures and orthopedic procedures) to determine an optimal plan. Aspects disclosed herein may be used in trauma cases to help determine a sequence of steps to be completed. Aspects disclosed herein may monitor steps of a procedure (e.g., surgical procedure) to determine how to optimize the procedure, including evaluating and/or optimizing indications, pre-operative evaluations, intra-operative evaluations, or post-operative evaluations. Aspects disclosed herein may improve surgical and non-surgical techniques, including medication types, timing of medication, and amounts of medication prescribed or otherwise used.

Digital twin systems discussed herein may provide guidance to organizations, such as a hospital or health care network an insurance company, or the government. The hospital may receive a variety of trend data from one or more digital twin systems, including data related to patient treatment plan compliance, surgeon technique, or treatment success rates for example.

While principles of the present disclosure are described herein with reference to illustrative embodiments for particular applications, it should be understood that the disclosure is not limited thereto. Those having ordinary skill in the art and access to the teachings provided herein will recognize additional modifications, applications, embodiments, and substitution of equivalents all fall within the scope of the embodiments described herein. Accordingly, the invention is not to be considered as limited by the foregoing description.

1-20. (canceled)

21. A computer-implemented method for generating and presenting guidance for treating a target patient with a musculoskeletal disease or musculoskeletal disorder, comprising:
receiving, by one or more processors, a plurality of prior data sets, including: i) at least one data set specific to the target patient from the patient, ii) at least one data set specific to the patient from a health care service provider including medical imaging data, and iii) at least one data set specific to a patient population with at least one common attribute with the target patient from a health care service provider;
executing an algorithm, stored in a non-transitory computer-readable storage medium, to cluster the target patient into a phenotype based on a similarity measure applied to the plurality of prior data sets, and to identify a pattern across the plurality of prior data sets associated with the phenotype, the pattern describing a probability of success for each of a plurality of potential treatment options for the target patient;
generating, by the one or more processors, a treatment pathway recommendation for the target patient based on the identified pattern;
generating and presenting an electronic display of the treatment pathway recommendation; and
transmitting the treatment pathway recommendation to one or more health care service providers over a network in real time, so that each of the one or more health care service providers has immediate access to the treatment pathway recommendation.

22. The computer-implemented method of claim 21, wherein the at least one data set specific to the target patient from the patient includes a patient reported outcome measure including a quality of life score or a forgotten joint score.

23. The computer-implemented method of claim 21, wherein the treatment pathway recommendation includes performing a radiological assessment, a magnetic resonance imaging (MRI) assessment, or a computerized tomography (CT) imaging assessment.

24. The computer-implemented method of claim 21, wherein the treatment pathway recommendation includes a non-surgical treatment plan, wherein the non-surgical treatment plan includes one or more rehabilitation exercises, one or more treatments from a health care provider, and/or one or more drug treatments.

25. The computer-implemented method of claim 21, wherein the treatment pathway recommendation includes a recommended implant and a recommended joint replacement surgical plan for a robotic medical procedure.

26. The computer-implemented method of claim 25, wherein the recommended joint replacement surgical plan is a full knee replacement plan or a partial knee replacement plan, and wherein the one or more processors generate a digital twin of the target patient including a mechanistic model of a biophysical representation of the the musculoskeletal anatomy of the target patient.

27. The computer-implemented method of claim 21, further comprising:
comparing, by the one or more processors, a digital twin of the target patient with a database of digital twins of other patients having one or more similar phenotypes to the target patient; and
executing an algorithm to determine whether a surgical procedure for the target patient is predicted to be more difficult than an average surgical procedure of the same type based on the comparison.

28. The computer-implemented method of claim 27, further comprising:
generating, by the one or more processors, a difficulty rating for the surgical procedure; and
adjusting a surgical schedule stored in a hospital database to incorporate additional time for the surgical procedure based on the difficulty rating.

29. The computer-implemented method of claim 21, wherein the at least one data set specific to the patient population includes robot data from a plurality of prior robotic surgical procedures, the robot data including a movement of a robotic arm during a surgical procedure for each of the patients within the patient population, and wherein the treatment pathway recommendation includes a recommended surgical plan for a robotic medical procedure including a recommended movement of a robotic tool to treat the target patient.

30. The computer-implemented method of claim 21, further comprising:
generating, by the one or more processors, a digital twin of the target patient, wherein the digital twin includes a mechanistic model of a biophysical representation of the musculoskeletal anatomy of the target patient;
comparing, by the one or more processors, the digital twin of the target patient with a database of digital twins of other patients having one or more similar phenotypes to the target patient;
executing an algorithm to determine whether a surgical procedure for the target patient is predicted to be more difficult than an average surgical procedure of the same type based on the comparison;
generating, by the one or more processors, a difficulty rating for the surgical procedure; and
adjusting a surgical schedule stored in a hospital database to incorporate additional time for the surgical procedure based on the difficulty rating.

31. A computer-implemented method for generating and presenting an electronic display of guidance for treating a target patient with a musculoskeletal disease or musculoskeletal disorder, comprising:
receiving, by one or more processors, a plurality of prior data sets, including: i) at least one data set specific to the target patient from the patient, ii) at least one data set specific to the patient from a health care service provider, and iii) at least one data set specific to a patient population with at least one common attribute with the target patient from a health care service provider, wherein the at least one data set specific to the patient population includes robot data from a plurality of prior robotic surgical procedures, the robot data including a movement of a robotic arm during a surgical procedure for each of the patients within the patient population;
executing an algorithm, stored in a non-transitory computer-readable storage medium, to identify a pattern across the plurality of prior data sets, the pattern describing a probability of success for each of a plurality of potential treatment options for the target patient;
generating, by the one or more processors, a treatment pathway recommendation for the target patient, wherein the treatment pathway recommendation includes a recommended surgical plan for a robotic medical procedure including a recommended movement of a robotic tool;
generating and presenting an electronic display of the treatment pathway recommendation; and
transmitting, to a robotic surgical system, instructions for movement of a robotic arm based on the recommended movement of the robotic tool, wherein the robotic surgical system executes the movement of the robotic arm to perform a joint replacement surgical procedure on the target patient.

32. The computer-implemented method of claim 31, wherein the at least one data set specific to the target patient from the patient includes post-operative data collected from the patient after a robotic surgical procedure, and wherein the treatment pathway recommendation includes a recommended post-operative rehabilitation exercise schedule for the target patient.

33. The computer-implemented method of claim 32, further comprising:
executing a second algorithm, stored in a non-transitory computer-readable storage medium, to identify a pattern across the plurality of prior data sets, the pattern describing a probability of success for each of a plurality of potential treatment options for the target patient;
generating, by the one or more processors, an updated treatment pathway recommendation for the target patient using the probability of success for each of a plurality of potential treatment options for the target patient; and
generating and presenting an electronic display of the updated treatment pathway recommendation, wherein the updated treatment pathway recommendation includes a recommended adjustment to the target patient's post-operative rehabilitation exercise schedule.

34. The computer-implemented method of claim 31, wherein the one or more processors generate a digital twin of the target patient, wherein the digital twin includes a mechanistic model of a biophysical representation of the musculoskeletal anatomy of the target patient.

35. The computer-implemented method of claim 34, further comprising:
comparing, by the one or more processors, the digital twin of the target patient with a database of digital twins of other patients having one or more similar phenotypes to the target patient; and
executing an algorithm to determine whether the joint replacement surgical procedure for the target patient is predicted to be more difficult than an average surgical procedure of the same type based on the comparison.

36. The computer-implemented method of claim 35, further comprising:
generating, by the one or more processors, a difficulty rating for the joint replacement surgical procedure; and
adjusting a surgical schedule stored in a hospital database to incorporate additional time for the joint replacement surgical procedure based on the difficulty rating.

37. The computer-implemented method of claim 31, wherein the at least one data set specific to the target patient from the patient is received via a wearable electronic device that monitors range of motion of a joint, and wherein the at least one data set specific to the patient from a health care service provider includes medical image data of the musculoskeletal anatomy of the target patient.

38. The computer-implemented method of claim 31, further comprising:
executing an algorithm to cluster the target patient into a phenotype based on a similarity measure applied to the plurality of prior data sets; and
transmitting the treatment pathway recommendation to one or more health care service providers over a network in real time, so that each of the one or more health care service providers has immediate access to the treatment pathway recommendation.

39. A computer-implemented method for generating treatment guidance using a digital twin system, comprising:
receiving, by one or more processors, patient data from a patient interface and a health care service provider, wherein the patient data includes medical image data of a target patient's musculoskeletal anatomy;
generating, by the one or more processors, a digital twin of the target patient, wherein the digital twin includes a mechanistic model of a biophysical representation of the target patient's musculoskeletal anatomy;
comparing, by the one or more processors, the digital twin of the target patient with a database of digital twins of other patients having one or more similar phenotypes to the target patient;
executing an algorithm to determine whether a surgical procedure for the target patient is predicted to be more difficult than an average surgical procedure of the same type based on the comparison;
generating, by the one or more processors, a difficulty rating for the surgical procedure; and
adjusting a surgical schedule stored in a hospital database to incorporate additional time for the surgical procedure based on the difficulty rating.

40. The computer-implemented method of claim 39, further comprising:
receiving, by the one or more processors, a plurality of prior data sets specific to a patient population with at least one common attribute with the target patient from a health care service provider;
executing an algorithm, stored in a non-transitory computer-readable storage medium, to cluster the target patient into a phenotype based on a similarity measure applied to the plurality of prior data sets; and
generating, by the one or more processors, a treatment pathway recommendation for the target patient based on the phenotype clustering.
